(19) United States
(12) Patent Application Publication
(10) Pub. No.: US 2026/0041664 A1
(43) Pub. Date: Feb. 12, 2026

(54) COMPOSITION AND USE THEREOF

(71) Applicant: NANJING NUTRABUILDING BIO-TECH CO., LTD., Nanjing (CN)

(72) Inventors: Long JIANG, Nanjing (CN); Ronghua yi, Nanjing (CN); Dawei Jin, Nanjing (CN); Shawn Wells, Frisco, TX (US); Kylin Liao, Nanjing (CN)

(21) Appl. No.: 19/297,204

(22) Filed: Aug. 12, 2025

(30) Foreign Application Priority Data
Aug. 12, 2024 (WO) PCT/CN2024/111413

(51) Int. Cl. A61K 31/357; A61K 31/121; A61P 1/16

(52) U.S. Cl. CPC A61K 31/357; A61K 31/121; A61P 1/16

(57) ABSTRACT

Provided is a method for improving or regulating liver metabolism level, enhancing or maintaining liver function, improving or maintaining liver health in a subject, which includes administering to the subject an effective amount of tetrahydrocurcumin and silymarin composition. The composition can be widely applied to nutritional supplements, foods and beverages.

### CROSS-REFERENCE TO RELATED APPLICATION

This application claims the priority benefit of International Patent Application No. PCT/CN2024/111413, filed on Aug. 12, 2024. The content of International Patent Application No. PCT/CN2024/111413 is incorporated herein by reference in its entirety and made a part of this specification.

### TECHNICAL FIELD

The present disclosure belongs to the field of biotechnology, and in particular relates to a method for improving or regulating liver metabolism level, enhancing or maintaining liver function, improving or maintaining liver health in a subject.

### BACKGROUND

Liver diseases cause more than 2 million deaths every year, accounting for 4% of the total number of deaths in the world. The prevalence of alcohol use disorder in the world is as high as 5.1%, and excessive drinking has become a global health problem. Alcohol can increase the mortality of liver diseases by 260 times, and the secondary alcoholic liver disease (ALD) is an important cause of morbidity and mortality of liver diseases. Alcoholic liver disease can initially manifest as fatty liver, and then it can develop into alcoholic hepatitis. Studies have pointed out that the prognosis and mortality of alcoholic hepatitis are far higher than expected, with the mortality rate of 14%-24% in 28 days and 56% in 5 years. Alcoholism for months to years can cause structural damage to the liver and ALD. In addition, ALD can usually coexist with other liver diseases, such as viral hepatitis and nonalcoholic fatty liver, and eventually develop into liver fibrosis and cirrhosis, and even liver cancer.

In the past decades, the number of cases of death due to ALD has been increasing continuously, covering various countries in Asia, Europe, America and Latin America. The research published in The Lancet, a top medical journal, points out that the onset of ALD is getting younger and younger, and the age is concentrated in the young adults aged 15-44, which brings more troubles and inconvenience to their lives. Protecting the liver has become the need of many sub-health people.

With the continuous improvement of people's concern about liver health, dietary health care products for protecting liver emerge in an endless stream. But the effect is not obvious, and the side effects are great. The inventor discovered for the first time that the composition of tetrahydrocurcumin (4HC) and silymarin and/or Silybum marianum (Sil) can protect liver.

### SUMMARY

In one aspect, the present disclosure provides a method for improving or regulating liver metabolism level, enhancing or maintaining liver function, improving or maintaining liver health in a subject, the method includes administering to the subject a composition including tetrahydrocurcumin and silymarin. In some embodiments, the method includes administering to the subject an effective amount of the composition.

In some embodiments, the improving or regulating liver metabolism level, enhancing or maintaining liver function, improving or maintaining liver health in a subject includes reducing or improving serum alanine aminotransferase (ALT), reducing or improving aspartate aminotransferase (AST), enhancing or improving antioxidant capacity of liver cells, relieving inflammation and apoptosis of liver cells, maintaining liver tissue morphology, reducing or preventing lesions.

In some embodiments, the administration amount and/or the effective amount of the composition is 0.1 mg-2000 mg or 0.1 μM to 1 M or 0.001 to 50.0 weight % (w/w). In some embodiments, the administration amount and/or the effective amount of the composition ranges from 0.1 mg to 2000 mg, 0.5 mg to 2000 mg, 1 mg to 2000 mg, 2 mg to 2000 mg, 0.5 mg to 1500 mg, 1 mg to 1500 mg, 5 mg to 1500 mg, 10 mg to 1200 mg, 20 mg to 1000 mg, 30 mg to 800 mg, 30 mg to 500 mg, 40 mg to 300 mg, 300 mg to 600 mg. In some embodiments, the administration amount and/or the effective amount of the composition ranges from 0.005% to 45%, 0.005% to 40%, 0.005% to 30%, 0.005% to 25%, 0.01% to 40%, 0.01% to 30%, 0.01% to 25%, 0.01% to 15%, 0.05% to 30%, 0.05% to 20%, 0.05% to 10%, 0.1% to 35%, 0.1% to 25%, 0.1% to 10%, 0.2% to 25%, 0.2% to 10%, 0.5% to 9%, 1% to 7%, 2% to 7%, 0.005% to 7%, 0.005% to 9% (w/w). In some embodiments, the administration amount and/or the effective amount of the composition ranges from 0.1 μM to 1 M (e.g., 0.1 μM to 500 μM, 1 PM to 500 μM, 1 μM to 5 mM, 1 μM to 500 mM, 5 μM to 500 μM, 5 μM to 5 mM, 5 μM to 100 mM, 5 μM to 500 mM, 50 μM to 500 μM, 50 μM to 5 mM).

In some embodiments, the ratio of tetrahydrocurcumin to silymarin is 1:500 to 500:1. In some embodiments, the ratio of tetrahydrocurcumin to silymarin may be 1:500 to 500:1, 1:300 to 300:1, 1:200 to 200:1, 1:100 to 100:1, 1:80 to 80:1, 1:50 to 50:1, 1:20 to 20:1, 1:10 to 10:1, 1:8 to 8:1, 1:8 to 6:1, 1:5 to 6:1, 1:5 to 4:1, 1:3 to 4:1, 1:3 to 3:1, 1:2 to 2:1, 1:6 to 41:1, 1:3 to 1:1, 1:2 to 1:1, or 1:1.

In some embodiments, the composition is prepared into solid preparation or liquid preparation. In some embodiments, the tetrahydrocurcumin and silymarin are prepared into solid preparation or liquid preparation.

In some embodiments, the composition is in the form of suppository, tablet, pill, granule, powder, film, capsule, beverage, aerosol, elixir, tincture, tonic, liquid suspension, syrup. In some embodiments, the tetrahydrocurcumin and silymarin are in the form of suppository, tablet, pill, granule, powder, film, capsule, beverage, aerosol, elixir, tincture, tonic, liquid suspension, syrup.

In some embodiments, the subject is a mammal. In some embodiments, the subject is human.

In some embodiments, the improving or regulating liver metabolism level, enhancing or maintaining liver function, improving or maintaining liver health in a subject is greater with administration of the composition including tetrahydrocurcumin and silymarin than without administration of the composition.

In some embodiments, the maintaining liver tissue morphology is greater with administration of the composition including tetrahydrocurcumin and silymarin than without administration of the composition.

In some embodiments, the reducing serum alanine aminotransferase (ALT), and aspartate aminotransferase (AST) is greater with administration of the composition including tetrahydrocurcumin and silymarin than without administration of the composition.

In one aspect, the present disclosure provides a composition including tetrahydrocurcumin and silymarin.

In some embodiments, the administration amount of the composition is 0.1 mg-2000 mg or 0.1 μM to 1 M or 0.001 to 50.0 weight % (w/w).

In some embodiments, the composition is administered at least once a day for 1 week to 7 weeks, 2 weeks to 7 weeks, 3 weeks to 7 weeks, or 4 weeks to 7 weeks.

In some embodiments, the composition can be administered by oral administration, intravenous injection, intramuscular injection, intraperitoneal injection, local drip or sublingual administration. For example, the composition is administered in a single dose or multiple divided doses. The daily dosage of the composition ranges from 0.1 mg to 2000 mg, 0.5 mg to 2000 mg, 1 mg to 2000 mg, 2 mg to 2000 mg, 0.5 mg to 1500 mg, 1 mg to 1500 mg, 5 mg to 1500 mg, 10 mg to 1200 mg, 20 mg to 1000 mg, 30 mg to 800 mg, 30 mg to 500 mg, 40 mg to 300 mg, 300 mg to 600 mg. In some embodiments, the daily dosage ranges from 0.005% to 45%, 0.005% to 40%, 0.005% to 30%, 0.005% to 25%, 0.01% to 40%, 0.01% to 30%, 0.01% to 25%, 0.01% to 15%, 0.05% to 30%, 0.05% to 20%, 0.05% to 10%, 0.1% to 35%, 0.1% to 25%, 0.1% to 10%, 0.2% to 25%, 0.2% to 10%, 0.5% to 9%, 1% to 7%, 2% to 7%, 0.005% to 7%, 0.005% to 9% (w/w). In some embodiments, the daily dosage ranges from 0.1 μM to 1 M (e.g., 0.1 μM to 500 μM, 1 μM to 500 μM, 1 μM to 5 mM, 1 μM to 500 mM, 5 μM to 500 μM, 5 μM to 5 mM, 5 μM to 100 mM, 5 μM to 500 mM, 50 μM to 500 μM, 50 M to 5 mM).

In some embodiments, the composition is used for improving or regulating liver metabolism level, enhancing or maintaining liver function, improving or maintaining liver health in a subject.

In some embodiments, the improving or regulating liver metabolism level, enhancing or maintaining liver function, improving or maintaining liver health in a subject includes reducing or improving serum alanine aminotransferase (ALT), reducing or improving aspartate aminotransferase (AST), enhancing or improving antioxidant capacity of liver cells, relieving inflammation and apoptosis of liver cells, and maintaining liver tissue morphology, reducing or preventing lesions.

In some embodiments, the ratio of tetrahydrocurcumin to silymarin is 1:10 to 10:1. In some embodiments, the ratio of tetrahydrocurcumin to silymarin may be 1:500 to 500:1, 1:300 to 300:1, 1:200 to 200:1, 1:100 to 100:1, 1:80 to 80:1, 1:50 to 50:1, 1:20 to 20:1, 1:10 to 10:1, 1:8 to 8:1, 1:8 to 6:1, 1:5 to 6:1, 1:5 to 4:1, 1:3 to 4:1, 1:3 to 3:1, 1:2 to 2:1, 1:6 to 41:1, 1:3 to 1:1, 1:2 to 1:1, or 1:1.

In some embodiments, the composition is prepared into solid preparation or liquid preparation.

In some embodiments, the composition is formulated as nutriment, dietary supplement, food, beverage or animal feed.

In some embodiments, the composition is in the form of suppository, tablet, pill, granule, powder, film, capsule, beverage, aerosol, elixir, tincture, tonic, liquid suspension, syrup.

In another aspect, the present disclosure provides use of tetrahydrocurcumin and silymarin in preparing a composition for improving or regulating liver metabolism level, enhancing or maintaining liver function, improving or maintaining liver health in a subject.

In some embodiments, the composition is used for preparing nutriment, dietary supplement, health product, food, beverage and animal feed. In nutriment, dietary supplement, food, beverage and animal feed, the weight ratio of the composition of the present disclosure is 0.1-95%.

In some embodiments, in nutriment, dietary supplement, food, beverage and animal feed, the weight ratio of the composition of the present disclosure is 1-50%.

In some embodiments, the improving or regulating liver metabolism level, enhancing or maintaining liver function, improving or maintaining liver health in a subject includes reducing or improving serum alanine aminotransferase (ALT), reducing or improving aspartate aminotransferase (AST), enhancing or improving antioxidant capacity of liver cells, relieving inflammation and apoptosis of liver cells, maintaining liver tissue morphology, reducing or preventing lesions.

In some embodiments, the ratio of tetrahydrocurcumin to silymarin is 1:10 to 10:1. In some embodiments, the ratio of tetrahydrocurcumin to silymarin may be 1:500 to 500:1, 1:300 to 300:1, 1:200 to 200:1, 1:100 to 100:1, 1:80 to 80:1, 1:50 to 50:1, 1:20 to 20:1, 1:10 to 10:1, 1:8 to 8:1, 1:8 to 6:1, 1:5 to 6:1, 1:5 to 4:1, 1:3 to 4:1, 1:3 to 3:1, 1:2 to 2:1, 1:6 to 41:1, 1:3 to 1:1, 1:2 to 1:1, or 1:1.

In some embodiments, the dosage of the composition is 0.1 mg-2000 mg or 0.1 μM to 1 M or 0.001 to 50.0 weight % (w/w). In particular, the daily dosage of the composition ranges from 0.1 mg to 2000 mg, 0.5 mg to 2000 mg, 1 mg to 2000 mg, 2 mg to 2000 mg, 0.5 mg to 1500 mg, 1 mg to 1500 mg, 5 mg to 1500 mg, 10 mg to 1200 mg, 20 mg to 1000 mg, 30 mg to 800 mg, 30 mg to 500 mg, 40 mg to 300 mg, 300 mg to 600 mg. In some embodiments, the daily dosage ranges from 0.005% to 45%, 0.005% to 40%, 0.005% to 30%, 0.005% to 25%, 0.01% to 40%, 0.01% to 30%, 0.01% to 25%, 0.01% to 15%, 0.05% to 30%, 0.05% to 20%, 0.05% to 10%, 0.1% to 35%, 0.1% to 25%, 0.1% to 10%, 0.2% to 25%, 0.2% to 10%, 0.5% to 9%, 1% to 7%, 2% to 7%, 0.005% to 7%, 0.005% to 9% (w/w). In some embodiments, the daily dosage ranges from 0.1 μM to 1 M (e.g., 0.1 μM to 500 μM, 1 μM to 500 μM, 1 μM to 5 mM, 1 μM to 500 mM, 5 μM to 500 μM, 5 μM to 5 mM, 5 μM to 100 mM, 5 μM to 500 mM, 50 μM to 500 μM, 50 μM to 5 mM).

### BRIEF DESCRIPTION OF THE DRAWINGS

FIG. 1 is the organ coefficient of liver of mice in different administration groups.

FIG. 2A-FIG. 2B show the effects of different administrations on serum ALT and AST activities in mice.

FIG. 3A-FIG. 3C are graphs showing the effects of different administrations on the activities of SOD, GSH and MDA in the liver of mice, respectively.

FIG. 4A-FIG. 4C show the effects of different administrations on the contents of TNF-α, IL-6 and caspase-3 in liver tissue of mice, respectively.

FIG. 5 shows the results of H&E staining and oil red O staining of liver tissues of mice in each group.

### DETAILED DESCRIPTION OF THE EMBODIMENTS

Reference will now be made in detail to the preferred embodiments of the present disclosure, examples of which are further illustrated. While the present disclosure will be described in conjunction with the preferred embodiments, it will be understood that they are not intended to limit the present disclosure to these embodiments. On the contrary, the present disclosure is intended to cover alternatives, modifications, and equivalents, which may be included within the spirit and scope of the present disclosure as defined by the claims. Furthermore, in the detailed description of the present disclosure, numerous specific details are set forth in order to provide a thorough understanding of the present disclosure. However, it will be obvious to one of ordinary skill in the art that the present disclosure may be practiced without these specific details. In other instances, well known methods, procedures, components, and other features have not been described in detail as not to unnecessarily obscure aspects of the present disclosure.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

As used herein, the terms “subject” or “individual” are used interchangeably to refer to any subject to whom the disclosed methods and compositions can be applied or administered. The subject may have a disease or disorder, but the subject does not need to be sick to benefit from the disclosed methods and compositions. Any subject can take the disclosed composition or become a recipient of the disclosed method. As used herein, the term “subject” refers to animals (for example, birds, reptiles and mammals). In some embodiments, the subject can be mammals including non-primates (e.g., camels, donkeys, zebras, cows, horses, cats, dogs, rats and mice) and primates (e.g., monkeys, chimpanzees and humans). In certain embodiments, the subject may be a non-human mammal. In other embodiments, the subject may be a human.

The term “administer”, “administering”, or “administration”, as used in the present disclosure, refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The contemplated method involves the daily administration of appropriate amounts of substances and compositions, depending on the formulation and form of the specific substances and compositions. This amount can be administered once or more per day. Typically, the effective amount for one or more times a day is 0.1 mg to 2000 mg. One or more doses can be administered once a day for any period of time. For example, an effective dose can be administered daily for one day, several days, many days or indefinitely, and the composition can be administered for 7 days or more in a period, depending on the desired effect. More typically, the daily administration amount ranges from 0.1 mg to 2000 mg, 0.5 mg to 2000 mg, 1 mg to 2000 mg, 2 mg to 2000 mg, 5 mg to 1500 mg, 10 mg to 1200 mg, 20 mg to 1000 mg, 30 mg to 800 mg, 30 mg to 500 mg, 40 mg to 300 mg, 300 mg to 600 mg. More typically, the amount administered per day is 5 mg to 1500 mg.

The technical features, implementation methods and beneficial effects of the present disclosure will be further described in detail with specific examples. The following examples are only part of the embodiments of the present disclosure, but not all of them. Unless otherwise specified, the materials and reagents described in the following examples are all common commercial products and can be purchased in the market.

### EXAMPLES

### Example 1

Compositions 1-15 were formulated according to the weight ratio shown in the following table:

| Composition | Tetrahydrocurcumin | Silymarin |
|---|---|---|
| 1 | 20 | 1 |
| 2 | 18 | 1 |
| 3 | 15 | 1 |
| 4 | 10 | 1 |
| 5 | 8 | 1 |
| 6 | 5 | 1 |
| 7 | 3 | 1 |
| 8 | 1 | 1 |
| 9 | 1 | 3 |
| 10 | 1 | 5 |
| 11 | 1 | 8 |
| 12 | 1 | 10 |
| 13 | 1 | 15 |
| 14 | 1 | 18 |
| 15 | 1 | 20 |

In addition to the example in Table 1, the composition of the present disclosure can be prepared according to appropriate content or content ratio.

### Example 2

Forty-two male C57 mice (6-8 weeks old) were raised under standard laboratory conditions (constant temperature 21±2° C., relative humidity 50-70% and alternating lighting scheme (12/12 hour light-dark ratio). All mice were free to drink water and eat, and adapted for 7 days. In order to prevent the influence of chronobiology on animals, the experiment was conducted at the same time every day (8 am to 12 am).

Six C57 mice were randomly selected from 42 mice as blank control group, and the remaining 36 mice were used as liver injury model group, and were given freely water solution containing 5% alcohol for 7 days.

On the 8th day, the blank control group was recorded as G0, and was still given water freely. Thirty-six liver injury model mice were randomly divided into 6 groups, with 6 mice in each group:
G1 (model group),
G2 (tetrahydrocurcumin group, H-4HC),
G3 (curcumin+silymarin group, M-HC+silymarin group),
G4 (low-dose tetrahydrocurcumin+silymarin group, L-4HC+silymarin group),
G5 (middle dose tetrahydrocurcumin+silymarin group, M-4HC+silymarin),
G6 (high dose tetrahydrocurcumin+silymarin group, H-4HC+silymarin).

On the 8th-24th day of the experiment, except for the control group (G0), other groups of mice continued to drink water solution containing 5% alcohol freely, and were administered by gavage according to the corresponding sample dosage of each group in Table 1. In addition, they were directly given water solution containing 10% alcohol (5 g/kg body weight) in the last 3 days of each week. This is an animal model, which combines continuous drinking with acute drinking, resulting in an increase in blood alcohol levels.

TABLE 1
Experimental Grouping

| | | | Amount (mg/kg) | |
|---|---|---|---|---|
| Group | Number | Sample | HC/4HC | Silymarin |
| G0 | 6 | Water | / | / |
| G1 | 6 | Water solution of alcohol | / | / |
| G2 | 6 | H-4HC | 55 | / |
| G3 | 6 | M-HC + silymarin | 20 | 60 |
| G4 | 6 | L-4HC + silymarin | 10 | 60 |
| G5 | 6 | M-4HC + silymarin | 20 | 60 |
| G6 | 6 | H-4HC + silymarin | 55 | 60 |

Mice were weighed and recorded weekly and on the last day of the experiment. Two hours after administration on the last day of the experiment, blood samples of each group of mice were collected. After blood collection, the animals were killed immediately, and the liver and kidney were taken and weighed, and the same liver tissue was extracted for histopathological examination and kit detection.

Body weight, liver weight and liver coefficient

According to the recorded body weight and liver weight, the trend chart was drawn, and the organ coefficient of the liver was calculated: liver coefficient (liver index)=organ weight/body weight×100%.

### Determination of Serum AST and ALT Levels

Blood was collected in a centrifuge tube and allowed to stand at room temperature for 1 h for serum chromatography. Then it was centrifuged at room temperature at 3000 rpm for 10 min, and the supernatant was transferred to a clean centrifuge tube. According to the manufacturer's instructions, the contents of ALT and AST in serum were determined with the corresponding detection kits. Detection of tumor necrosis factor (TNF-α), interleukin-6 (IL-6), superoxide dismutase (SOD), glutathione (GSH), malondialdehyde (MDA) and apoptosis (Caspase-3, a biomarker of hepatocyte apoptosis)

The same part of the liver tissue of each mouse was accurately weighed, and then homogenized with PBS according to the mass/volume ratio of 1:9. After centrifugation at 3000 g for 15 minutes, the supernatant was collected, and the contents of each index in the liver homogenate were measured by using corresponding kits according to the manufacturer's instructions.

### Examination of Liver Histopathology

The same part of liver tissue of each mouse was fixed with 4% paraformaldehyde solution, and then paraffin-embedded sections were made. The sections were stained with hematoxylin-eosin (H&E staining) and oil red O staining respectively, and observed under the optical microscope.

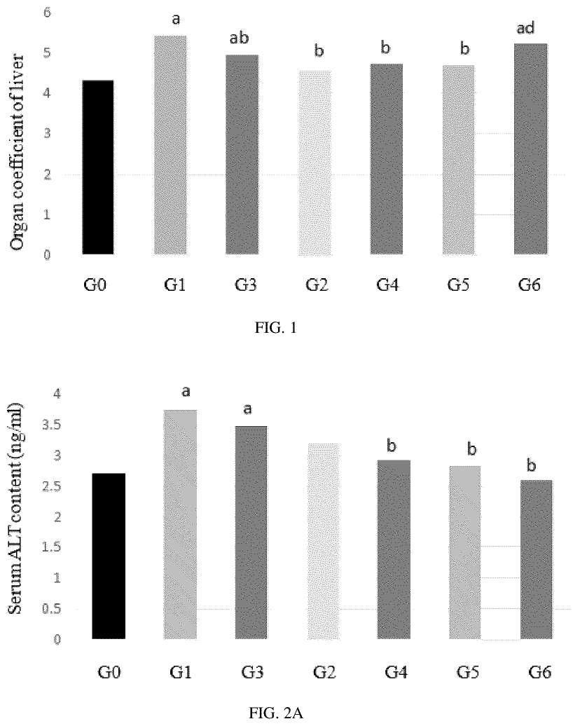

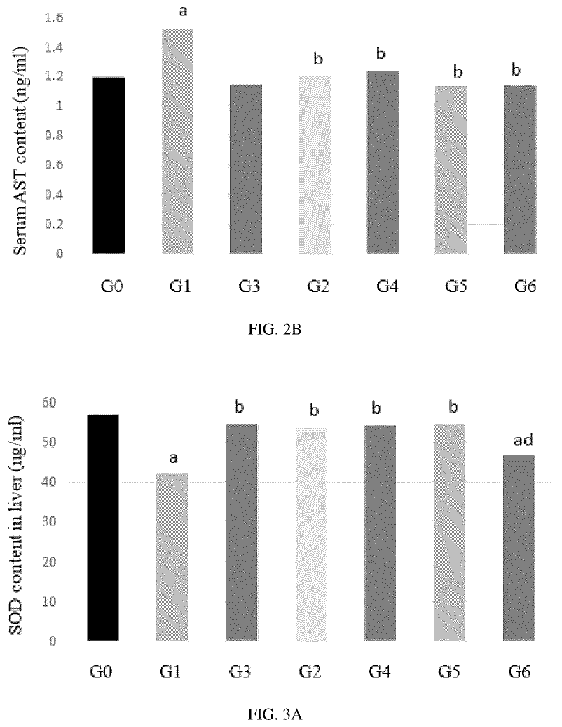

Effects of different administration treatments on serum ALT and AST contents in mice All kinds of external liver stimulation can cause the increase of serum ALT and AST activities. ALD (alcoholic liver disease) can cause significant liver injury, thus releasing ALT and AST from liver cells into serum, and greatly increasing the levels of ALT and AST in serum, among which alcohol has greater influence on AST release than on ALT. FIG. 1 is the organ coefficient of liver of mice in different administration groups. FIG. 2A-FIG. 2B show the effects of different administrations on serum ALT and AST activities in mice. a stands for significant difference compared with control group, p<0.05; b means that there is a significant difference between each administration group and alcohol treatment group, p<0.05; c indicates that there is a significant difference between M-HC+silymarin and M-4HC+silymarin, p<0.05; d indicates that there is a significant difference between H-4HC and H-4HC+silymarin, p<0.05. In the experiment, it was found (FIG. 1 and FIG. 2) that the liver-to-weight ratio, serum ALT and AST levels of all drinking mice receiving dietary supplements decreased to some extent compared with those drinking only. The liver coefficient of G5 (M-4HC+silymarin) is 5% lower than that of G3 (M-HC+silymarin). The combination of M-4HC and silymarin has the best effect, which can significantly reduce the ALT and AST levels of drinking mice by 24.6% and 25.2% respectively. The ALT and AST levels of G5 (M-4HC+silymarin) are 18.6% and 0.45% lower than that of G3 (M-HC+silymarin), respectively.

### Effects of Different Administration Treatments on SOD Activity, GSH and MDA Content in Liver of Mice

In the process of alcohol metabolism, the liver will produce high levels of reactive oxygen species (ROS), which will rapidly consume antioxidant substances such as glutathione (GSH) in cells. In this process, activity of superoxide dismutase (SOD) will also be inhibited. The increase of oxidative stress is closely related to the pathogenesis of alcoholic liver disease (ALD). For example, polyunsaturated fatty acids are peroxidized and degraded to produce malondialdehyde (MDA), which is further crosslinked with lipoprotein to produce cytotoxicity and further damage to the liver. These are common indicators for liver disease detection and judgment.

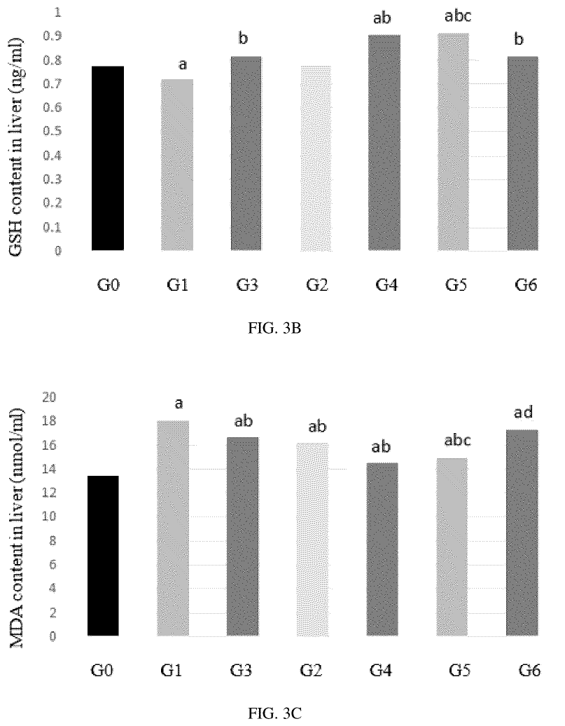

FIG. 3A-FIG. 3C are graphs showing the effects of different administrations on the activities of SOD, GSH and MDA in the liver of mice, respectively. a stands for significant difference compared with control group, p<0.05; b means that there is a significant difference between each administration group and alcohol treatment group, p<0.05; c indicates that there is a significant difference between M-HC+silymarin and M-4HC+silymarin, p<0.05; d indicates that there is a significant difference between H-4HC and H-4HC+silymarin, p<0.05. It can be seen from FIG. 3A-FIG. 3C that the intervention of 4HC+silymarin combination can significantly increase the GSH level and decrease the MDA level in the liver of drinking mice, and its effect is statistically different from that of the same dose of HC+silymarin combination. The effect of M-4HC+silymarin combination is still the best, and the GSH level is 27% higher than that of drinking model mice and 18.1% higher than that of normal mice. The GSH level in M-HC+silymarin group is 14% higher than that in drinking model mice and 5.3% higher than that in normal mice. The GSH level in H-4HC group is 7.9% higher than that in drinking model mice and 0.3% higher than that in normal mice. The GSH level of G5 (M-4HC+silymarin) is 12.04% higher than that of G3 (M-HC+silymarin). The MDA level of M-4HC+silymarin group was 16.8% lower than that of drinking model mice, and that of M-HC+silymarin group was 7% lower than that of drinking model mice, and that of H-4HC group was 9.3% lower than that of drinking model mice. The MDA level of G5 (M-4HC+silymarin) is 10.43% lower than that of G3 (M-HC+silymarin). At the same time, SOD activity was not significantly inhibited, which was close to that of normal mice. The SOD content of G5 (M-4HC+silymarin) is 0.24% lower than that of G3 (M-HC+silymarin).

### Effects of Different Administration Treatments on Tumor Necrosis Factor-α (TNF-α), Proinflammatory Factor Interleukin-6 (IL-6) and Apoptosis (Caspase-3) in Mice Liver

Alcohol can increase the levels of many inflammatory factors in liver cells. Like the oxidative stress brought by ROS, inflammation is also an important cause of ALD. Some studies have pointed out that the increase of pro-inflammatory factors TNF-α and IL-6 is related to the degree of liver damage caused by alcohol and the occurrence and development of ALD. Due to the double stimulation of inflammation and oxidative stress, the activity of apoptosis factors in hepatocytes, such as Caspase-3, often increases, which can eventually lead to hepatocyte apoptosis.

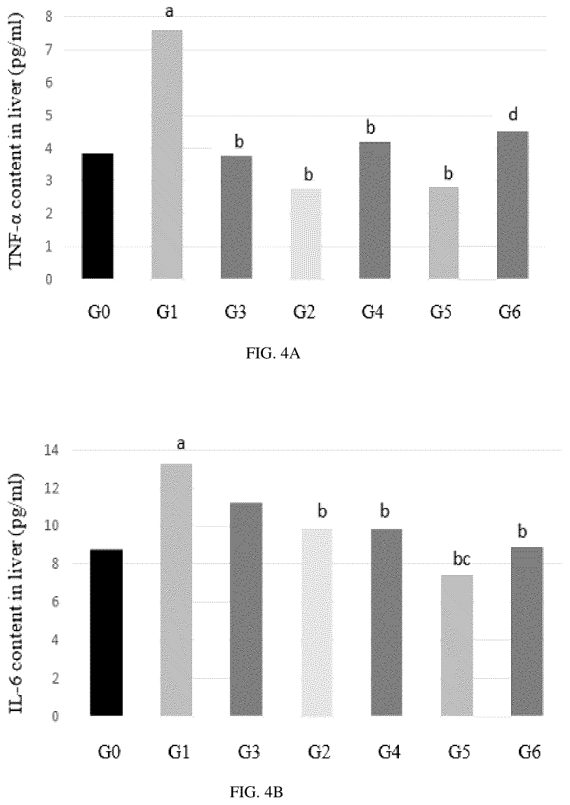

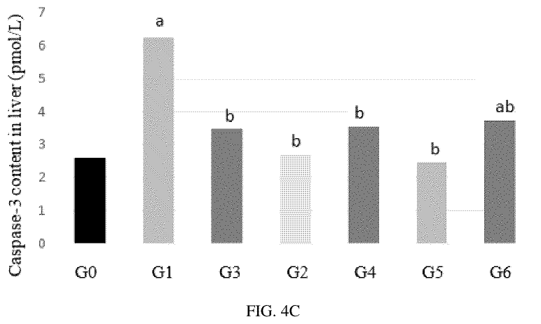

The experimental study found that the liver inflammatory factors and apoptosis factors of drinking mice decreased significantly after receiving dietary supplement intervention. FIG. 4A-FIG. 4C show the effects of different administrations on the contents of TNF-α, IL-6 and caspase-3 in liver tissue of mice, respectively. a stands for significant difference compared with control group, p<0.05; b means that there is a significant difference between each administration group and alcohol treatment group, p<0.05; c indicates that there is a significant difference between M-HC+silymarin and M-4HC+silymarin, p<0.05; d indicates that there is a significant difference between H-4HC and H-4HC+silymarin, p<0.05. Regarding TNF-α level, the M-4HC+silymarin combination still performed well, which decreased by 63%, while M-HC+silymarin combination decreased by 52.5%. The TNF-α level of G5 (M-4HC+silymarin) is 24.86% lower than that of G3 (M-HC+silymarin). Regarding IL-6 level, M-4HC+silymarin combination still performed well, which decreased by 44.1%, while M-HC+silymarin combination decreased by 15.75%. The IL-6 level of G5 (M-4HC+silymarin) is 33.82% lower than that of G3 (M-HC+silymarin). Regarding Caspase-3 level, M-4HC+silymarin combination still performed well, which decreased by 60.6%, while M-HC+silymarin combination decreased by 43.28%. The Caspase-3 level of G5 (M-4HC+silymarin) is 29.01% lower than that of G3 (M-HC+silymarin). Effects of different administrations on liver histopathology in mice

Typical damages of alcohol to liver tissue include steatosis, fused parenchymal necrosis, collagen, cystic degeneration in sinus and around the center, lobular inflammation affecting the area around veins, and foamy degeneration of liver cells. Severe cases may include perivenous and pericellular fibrosis, which indicates future cirrhosis.

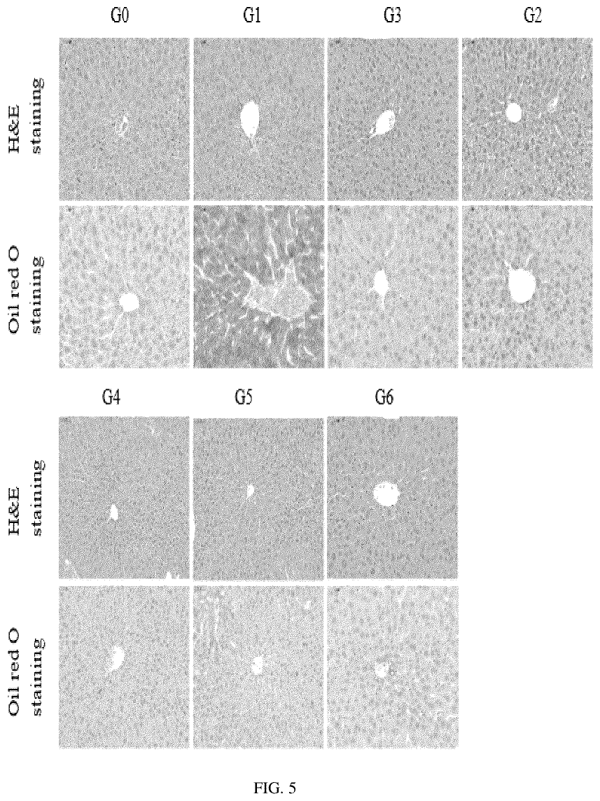

The results of H&E staining and oil red O staining shows that the central vein in the liver tissue of normal mice is clear, the structure of hepatic cord and hepatic sinus is normal, and there is no hepatic steatosis. Compared with normal mice, the liver cords of drinking mice are not arranged in a regular way, and there are a lot of vacuoles in the liver cells, which are accompanied by severe steatosis, and the liver tissue has suffered moderate damage. FIG. 5 shows the results of H&E staining and oil red O staining of liver tissues of mice in each group. After the intervention of dietary supplements, the damage degree of liver tissue in drinking mice has been improved to some extent. Consistent with the above results, M-4HC+silymarin combination significantly improves the disorder of hepatic cord and steatosis of hepatocytes, and the cytoplasm of hepatocytes is more uniform. L-4HC+silymarin combination also has a good effect on alleviating liver damage. Whereas, the traditional M-HC+silymarin combination and H-4HC can alleviate the changes of liver tissue, but the effect is poor.

The above are only the preferred embodiments of the present disclosure, and are not intended to limit the present disclosure. Those skilled in the art can make many changes, modifications, substitutions and variations on these embodiments without departing from the principles and purposes of the present disclosure, and the scope of the present disclosure is defined by the claims and their equivalents.

What is claimed is:

1. A method for improving or regulating liver metabolism level, enhancing or maintaining liver function, improving or maintaining liver health in a subject, comprising administering to the subject a composition comprising tetrahydrocurcumin and silymarin.

2. The method according to claim 1, wherein the improving or regulating liver metabolism level, enhancing or maintaining liver function, improving or maintaining liver health in a subject comprises reducing or improving serum alanine aminotransferase (ALT), reducing or improving aspartate aminotransferase (AST), enhancing or improving antioxidant capacity of liver cells, relieving inflammation and apoptosis of liver cells, and maintaining liver tissue morphology.

3. The method according to claim 1, wherein the composition is administered in an amount of 0.1 mg to 2000 mg, or 0.1 μM to 1 M, or 0.001 weight % (w/w) to 50.0 weight % (w/w).

4. The method according to claim 1, wherein a ratio of the tetrahydrocurcumin to the silymarin is 1:500 to 500:1.

5. The method according to claim 1, wherein a ratio of the tetrahydrocurcumin to the silymarin is 1:10 to 10:1.

6. The method according to claim 1, wherein the composition is prepared into a solid preparation or a liquid preparation.

7. The method according to claim 1, wherein the composition is in a form of suppository, tablet, pill, granule, powder, film, capsule, beverage, aerosol, elixir, tincture, tonic, liquid suspension, or syrup.

8. The method according to claim 1, wherein the subject is a mammal.

9. The method according to claim 1, wherein the improving or regulating liver metabolism level, enhancing or maintaining liver function, improving or maintaining liver health in a subject is greater with administration of the composition comprising the tetrahydrocurcumin and the silymarin than without administration of the composition.

10. The method according to claim 2, wherein the maintaining liver tissue morphology is greater with administration of the composition comprising the tetrahydrocurcumin and the silymarin than without administration of the composition.

11. The method according to claim 2, wherein at least one of the reducing serum alanine aminotransferase (ALT) or the reducing aspartate aminotransferase (AST) is greater with administration of the composition comprising the tetrahydrocurcumin and the silymarin than without administration of the composition.
